(19) United States
(12) Patent Application Publication
(10) Pub. No.: US 2021/0106784 A1
(43) Pub. Date: Apr. 15, 2021

(54) Face Cradle Cover with Immediate Essential Oil Decongestant Element

(71) Applicant: Dwayne Haynes, Fort Washington, MD (US)

(72) Inventor: Dwayne Haynes, Fort Washington, MD (US)

(21) Appl. No.: 16/912,699

(22) Filed: Jun. 25, 2020

Related U.S. Application Data
(60) Provisional application No. 62/866,271, filed on Jun. 25, 2019.

(51) Int. Cl. A61M 21/02; A61G 13/00; A61G 13/12

(52) U.S. Cl. CPC A61M 21/02; A61G 13/009; A61M 2021/0016; A61M 2210/0606; A61G 13/121

(57) ABSTRACT

The present invention relates to a cover for a massage table face cradle. Specifically, the invention is a fitted face cover for a massage table that further adds an immediate essential oil decongestant element for its user, providing a protective sanitary barrier, and an easy essential oil breathing experience while said user is in a prone (lying face down) position while receiving massage.

### CROSS-REFERENCE TO RELATED APPLICATIONS:

APPLICATION NUMBER: US 62/866,271

FILING DATE: Jun. 25, 2019

### BACKGROUND OF THE INVENTION:

A standard massage table can be generally described as having two exemplary main portions: the table top and the embodiment of the face cradle insert. The design of a massage table often includes a gap that separates the table top from the face cradle. This gap, including the hardware with connecting brackets that attach to the face cradle to the table top, provides a place for a person to place their face and head during the massage. It is standard practice in the massage industry to clean the massage table between customers, and provide clean linens and covers for the face cradle insert.

Essential oils have been used in forms of alternative medicine since the ancient Egyptians times, 4500 B.C.1 The oils became more recently popular in the late 70's and 80s for healing remedies and later to replace harmful chemicals in the home. Recently, its vast benefits have been vetted and they are now a staple in the massage industry. Massage therapists generally have three basic methods of administering these oils. The three methods include: 1. aroma diffusers, 2. adding essential oils to a carrier oil (e.g. fractured coconut oil, jojoba oil) then applying it directly to a client's skin, or 3. adding oils to hand for a three-breathe inhalation method before massage begins. All these methods are great, but the oils work best on an individual's brain's olfactory sense which is related with the sense of smell.

Although all methods of administering essential oils are seemingly practical, each method of dispersing the oils has limitations. First, the aroma diffuser can lose its potency while in use just moments after placing the oils inside the diffuser. The essential oil particles are wasted in the air rather directly impacting the olfactory sense. Not to mention, the diffuser may leave behind for future clients, particles in the spa space that may cause some unwanted or potential allergic scents. The second method, adding essential oil drops to a carrier oil like coconut or jojoba, is a better method as the oils can make their way into the integumentary system efficiently, but can be a costly method for spa owners. One 100% grade 15ml essential oil bottle can cost upwards of $25.00 per bottle, with each bottle holding approximately 200 drops per bottle. Some therapists use upwards of 8-12 drops per session which would give the 15 ml bottle a 25-session life period. Conversely, if a therapist used the Face Cradle Cover with Immediate Essential Oil Decongestant Element, only 2 drops of essential oil are needed per 60-90 minute session. The last method includes the client face down in the traditional “U-shaped” face cradle where there is an open breathing hole for the user. A therapist would normally then place a couple of essential oil drops in their hand, then instruct the client to take 3 breaths of their hands through the breathing hole. For this method, the Face Cradle Cover acts as the hands of the therapist for the duration of the client's massage. Instead of 3 breaths of the oil, the client is now infused with the oil in with every breath as the therapist focuses on the act of massaging. Clearly the spa gets a practical, safe, and effective method to administer essential oil and decongest the client while saving money on drops used per session by using the Face Cradle Cover with Immediate Essential Oil Decongestant Element.

### FIELD OF THE INVENTION

The present invention relates to a cover for a massage table face cradle cover. It provides a protective sanitary barrier, and an easy essential oil breathing experience while said user is in a prone position while receiving massage. Distinctively, the invention is a fitted face cradle cover for a massage table face cradle insert that delivers a prompt essential oil decongestant element for the user.

### BRIEF SUMMARY OF THE INVENTION

The present invention provides a cover for a face cradle on a massage table that is further configured with an immediate essential oil decongestant element for clients who are prone during a massage. This is achieved by maximizing the essence of the traditional “U-shaped” cover which is to provide a protective sanitary barrier for the user and the massage table.

An embodiment of the present invention is a face cradle cover that provides a sanitary surface for the user, with the addition of a portion allowing for an immediate essential oil decongestant element. This hanging “drum-like” feature just below the face cradle portion is composed of two portions. One portion houses a Velcro portion which serves as an essential oil entry position. The second portion houses a zipper portion which serves as a client essential oil/ fresh air control unit that is user adjustable to their respiratory specific needs. The inside of the hanging “drum-like” feature is coated with a terry cloth lining to help keep the essential oil scent that clients breathe. The hanging “drum-like” portion connects to the face cradle portion via ¾ permanent semi-circle stitch to the top the hanging “drum-like” portion. The ¾ permanent semi-circle stitch forms below the traditional “U-shaped” cover form which closes the traditionally opened breathing hole. All these features form the face rest cradle cover with an immediate essential oil decongestant element.

According to an embodiment of the present invention, reveals a face cradle cover with two portions, one for sanitary purposes, and one hanging “drum-like” feature with two partitions, one being a Velcro partition for essential oil entry, and the other a zipper partition for client fresh oxygen/essential oil intake. Which but portions help to create the immediate essential oil decongestant system.

According to an embodiment of the present invention, reveals the top “u-shaped” portion which serves as a protection to the exposed massage table, and serves as a sanitary barrier for the user.

According to an embodiment of the present invention, the “U-shaped” portion of the apparatus is composed of industry leading materials to universally fit massage headrest inserts. The “u” shaped portion is composed of materials to create a comfortable prone (face down) position for its user, and serves as protection to the massage table and adjacent areas.

According to an embodiment of the present invention, the “U-shaped” portion of the apparatus is composed of specialized threads, stitching, and material.

According to an embodiment of the present invention, the “U-shaped” portion threads, stitches, and material create a snug fit to envelope the face cradle insert.

According to an embodiment of the present invention, the hanging “drum-like” portion utilizes those same specialized threads and stitches to create a connection to the base of the face cradle cover portion.

According to an embodiment of the present invention, the hanging “drum-like” portion is a hollowed hanging “drum-like” portion that consists of two partitions.

According to an embodiment of the present invention, the hanging “drum-like,” portion consists of two partitions: one being a Velcro partition, and the other a zipper partition.

According to an embodiment of the present invention, the Velcro partition is located near the bottom of the apparatus front view, for essential oil entry.

According to an embodiment of the present invention, the zipper partition is located mid-seam of the apparatus front view, for client controlled oxygen/essential oil air intake.

According to an embodiment of the present invention, a hanging “drum-like” shaped portion with both its partitions is hollowed out with an internal lining (terry cloth).

According to an embodiment of the present invention, the terry cloth lined in the hanging “drum-like” shaped portion of the apparatus is used to lock in the essential scent, while recirculating clients breathe to create the decongestant system portion of the apparatus.

According to an embodiment of the present invention, the hanging “drum-like” portion of the apparatus consisted of the Velcro, and the zipper partition, which also includes a nasal opening created via the ¾ sealed connection to the face cradle cover. With the client's ultimate control via the zipper partition, they can control their own decongestant element while prone during massage. The apparatus acts as a “sophisticated paper bag” used to calm anxiety attacks, which the face cradle cover performs in the same manner to ease the users breathing with their choice essential oil.

The foregoing summary of the present invention with the preferred embodiments should not be construed to limit the scope of the invention. It should be understood and obvious to one skilled in the art that the embodiments of the invention thus described may be further modified without departing from the spirit and the scope of the invention.

### BRIEF DESCRIPTION OF THE DRAWINGS:

FIG. 1 is a front closed view of the face cradle cover with an immediate essential oil decongestant element in accordance with an embodiment of the present invention

FIG. 2 is a top view of the face cradle cover with an immediate essential oil decongestant element in accordance with an embodiment of the present invention

FIG. 3 is a backside view of the face cradle cover with an immediate essential oil decongestant element in accordance with an embodiment of the present invention

FIG. 4 is a side opened view of the face cradle cover with an immediate essential oil decongestant element in accordance with an embodiment of the present invention

FIG. 5 is a front closed view of the face cradle cover in accordance with an embodiment of the present invention.

### DETAILED DESCRIPTION OF THE INVENTION:

The present invention relates to a cover for a massage table face cradle cover. Specifically, the invention is a fitted cover for a massage table face cradle that further includes a hanging “drum-like” feature to comprise an immediate essential oil decongestant system that also serves as a protectant barrier for the massage table and all adjacent surface areas.

According to an embodiment of the present invention, a face cradle cover with an immediate essential oil decongestant element, is primarily of a face cradle cover portion and an essential decongestant element that hangs below the cover portion. In an alternate embodiment, the face cradle cover with an immediate essential oil decongestant element may comprise fewer or additional components. One of ordinary skill in the art would appreciate that a face cradle cover with an immediate essential oil decongestant element could be composed of a variety of components, and embodiments of the present invention are contemplated for use with any such component.

According to an embodiment of the present invention, a face cradle cover with an immediate essential oil decongestant element is primarily configured to serve as a hygienic cover that is placed over the face cradle insert of a massage table. In preferred embodiment, the face cradle cover portion is primarily adapted to wrap around and envelope the face cradle insert while in use on the massage table. In the preferred embodiment, the face cradle cover with immediate essential oil decongestant element is configured to drape over the front edge of the massage table top, as well as any hardware that supports the connection to the face cradle insert of the massage table. The immediate essential oil decongestant element may also cover any gap that exists between the front edge of the table top of the massage table and the back edge of the face cradle insert. In this way, the combination of the face cradle cover portion and the immediate essential oil decongestant element function together to prevent the face, eyes, nose, and/or mouth of a user from coming into direct contact with the massage table's surfaces. In preferred embodiment, the immediate essential oil decongestant element may be further configured to create a less congesting space for the user. One, of ordinary skill in the art, would appreciate that the face cradle cover with an immediate essential oil decongestant element could be adapted to function in a variety of forms and factors; embodiments of the present invention are contemplated for use with any such form factor.

According to an embodiment of the present invention, a face cradle cover with an immediate essential oil decongestant element is primarily constructed of fabric material. In a preferred embodiment, the face cradle cover with immediate essential oil decongestant element may be made from any soft durable fabric. The fabric may be a natural material such as cotton, or synthetic material, such as acrylic. In an alternate embodiment, the fabric may be an antibacterial or antimicrobial fabric or a fabric that is treated with an antibacterial or antimicrobial finish. In still further embodiments, the face cradle cover could be made from fabrics or other materials that are washable and reusable. Further embodiments may be constructed from disposable materials (e.g. plastic, latex). One of ordinary skill in the art would appreciate that there are a number of materials and fabrics that would be suitable for constructing the face cradle cover with an immediate essential oil element, and the embodiments of the present invention are contemplated for use with any such materials or fabric.

According to an embodiment of the present invention, the face cradle cover portion with essential oil decongestant element comprises a face cradle cover portion. In preferred embodiment, the face cradle cover portion consists primarily of a cover wall, one or more cover wall securing means, and one hanging “drum-like” feature hung directly beneath its ¾ semi circle permanent stitch seal. In an alternate embodiment, the face cradle cover portion may be composed of additional or fewer components. In preferred embodiments, the face cradle cover portion is adapted to surround and cover the face cradle of the massage table in order to provide a protective, hygienic barrier between the face and/ or head or a user and the surface or the face cradle. In some embodiments, the face cradle cover may be loosely fitted to provide a universal fit with a number of different face cradle inserts. One of ordinary skill in the art would appreciate a face cradle cover that could be designed in a number of configurations, and embodiments of the present invention are contemplated for use with any such configurations.

According to an embodiment of the present invention, the face cradle cover portion comprises a cover wall. In a preferred embodiment, the cover wall is one or more sections of material or fabric that are adapted to envelop the face cradle of a massage table. In the preferred embodiment, the cover wall may take the form of a shell or sleeve-like component that includes one or more openings that are adapted to allow the cover wall to slide on or over the face cradle of the massage table. In the preferred embodiment, the cover wall may comprise a single section of material that is appropriately formed and designed to be sewn, fused, sealed, or otherwise connected to form the cover wall. In an alternate preferred embodiment, the cover wall may comprise a plurality of sections of material that are each appropriately formed and designed to be sewn, fused, sealed or otherwise connected together to construct a cohesive unit that forms a cover wall. One of ordinary skill in the art would appreciate that there are many suitable design configurations for a cover wall, and the embodiments of the present invention are contemplated for use with any such design configuration.

According to an embodiment of the present invention, the face cradle cover portion comprises one or more cover wall securing means. In a preferred embodiment, the cover wall securing means is attached at the one or more openings on the cover wall. In the preferred embodiment, the cover wall securing means may cause the cover wall to substantially enclose the face cradle by forming a shell around the portions of the face cradle that would come into contact with the face and/or head of a user. In an alternative preferred embodiment, the cover wall securing means may cause the cover wall to fully enclose the face cradle by forming a fully scaled sleeve that completely covers the face cradle. In preferred embodiments, a number of items could be used as cover wall securing means, including but not limited to elastic elements, zippers, claps, buttons, hook- and loop fasteners, snap closures, magnets, or any other suitable connectors, or flexible fitting element. One of ordinary skill in the art would appreciate that there are a number of items that could be suitably used as a cover wall securing means, and embodiments of the present invention are contemplated for use with any such item. Further one of ordinary skill in the art would appreciate that the securing means could be formed, integrated or secured on any side or portion of the face cradle cover (e.g. top, bottom, left side, right side, front, and/or back) and these embodiments of the present invention are contemplated securing means.

According to an embodiment of the present invention, the face cradle cover is composed of an immediate essential oil decongestant element. In a preferred embodiment, the immediate essential oil decongestant element is primarily formed as a hanging “drum-like” attachment to the face cradle cover portion attachment means. In an alternate embodiment, the immediate essential oil decongestant element may comprise additional or fewer components. In preferred embodiments, the immediate essential oil decongestant element is adapted to cover areas of the massage table that are directly sewn below the face cradle cover portion in order to provide a protective, hygienic barrier between the face and/or head of a user and, in particular, the front edge of the massage table top, as well as other exposed portions of the massage table and face cradle in that area. One of ordinary skill in the art would appreciate that an immediate essential oil decongestant element could be designed in a number of configurations, and embodiments of the present invention and are contemplated for use with any such configurations.

According to an embodiment of the present invention, the immediate essential oil decongestant element is a hanging “drum-like” feature that is adapted to hang just beneath the cradle cover portion. In the preferred embodiment, the hanging “drum-like” feature has three primary purposes, each which has been previously discussed but are described again for clarity. The first portion houses the Velcro partition serving as an access point for essential oil drops scents via tissue paper. The second housing the zipper partition serves as a client/user essential oil/oxygen air intake access point to alleviate congestion in the hollowed terry cloth lined inside. Lastly, the immediate essential oil decongestant element serves as a protective function in that it serves as a cover barrier that envelops over the exposed portions of the massage table face cradle cover insert. In an additional preferred embodiment, the immediate essential oil decongestant element serves as a protection function. In addition, the bottom allows for a therapist to add a desired amount of essential oil drops to a tissue paper (and/or but not limited to cotton balls, or other aroma scented products) to add to the users massage experience. One of ordinary skill in the art would appreciate that there are many functions and configurations that the immediate essential oil decongestant element might embody, and the embodiments of the present invention are contemplated for use with any such function or configuration.

According to an embodiment of the present invention, the immediate essential oil decongestant element consists of one or more cover portion attachment means. In a preferred embodiment, in one portion attachment means is fixed to the surface directly beneath the face cradle cover portion to form the entire apparatus. In the preferred, one cover portion attachment means is configured to connect the face cradle portion to the immediate essential oil decongestant element. In the preferred embodiment, a variety of connectors may be used to connect the immediate essential oil decongestant element to the face cradle cover portion, (threads, stitches) but not limited to any lead industry materials. One of ordinary skill in the art would appreciate a number of connectors suitable to use a cover portion attachment means and embodiments of the present invention that are contemplated for use with any such connector. Further, one of ordinary skill in the art would appreciate the cover portion attachment means formed, integrated or secured on any side or portion of the face cradle cover (e.g., top, bottom, left , right side, front or back) and embodiments of the present invention are contemplated for use with all configurations of the cover portion attachment means.

According to an embodiment of the present invention, the immediate essential oil decongestant element is connected to the face cradle cover portion. In the preferred embodiments, there are many alternatives as to how this connection is primarily achieved by connecting the top end of the immediate essential oil decongestant element into the bottom off the face cradle cover portion of the face cradle cover element. In a first preferred embodiment, the immediate essential oil decongestant element is ¾ semi-circle sewn permanently connected to the bottom portion of the face cradle cover. One of ordinary skill in the art would appreciate that there are many methods that might be used to connect the immediate essential oil decongestant element to the face cradle cover portion, and the embodiments of the present invention are contemplated for use with any such connection methods.

According to an embodiment of the present invention, the immediate essential oil decongestant element may further include a protective lining on the interior wall of the immediate essential oil decongestant element. The first purpose of the interior lining serves as a double coating to aid locking in the essential oils scent inside the immediate essential oil decongestant element. The second purpose is to aid warming the user's nasal passages to allow the nasal passages to clear while prone during massage. In preferred embodiments, the protective lining could be formed from many materials such as fabric, linen vinyl, or any other lead industry materials. One of ordinary skill in the art would appreciate that there are numerous materials, and manners in which the protective lining could be second to form this portion of the immediate essential oil decongestant element, and embodiments of the present invention are contemplated for use with any such material or configuration.

### Exemplary Embodiments

According to an embodiment of the present invention, the face cradle cover with immediate essential oil decongestant element is adapted to envelop the face cradle cover insert. In a preferred embodiment, the face cradle cover portion of the immediate essential oil decongestant element is slipped over the face cradle insert of a massage table. Once the face cradle cover portion is in the place of the face cradle cover portion of the massage table, the cover wall securing means of the face cradle cover will securely hold the face cradle cover portion in place on the face cradle. In some embodiments, the cover wall securing means will be substantially secured by an elastic thread element that will be appropriate for the massage table and therapist. In preferred embodiment, the immediate essential oil decongestant element is permanently attached (¾ semi-circle sewn) below the face cradle cover portion. In preferred embodiment, the protective barrier (face cradle cover) and the hanging “drum-like” feature element forms the face cradle cover with an immediate essential oil decongestant element.

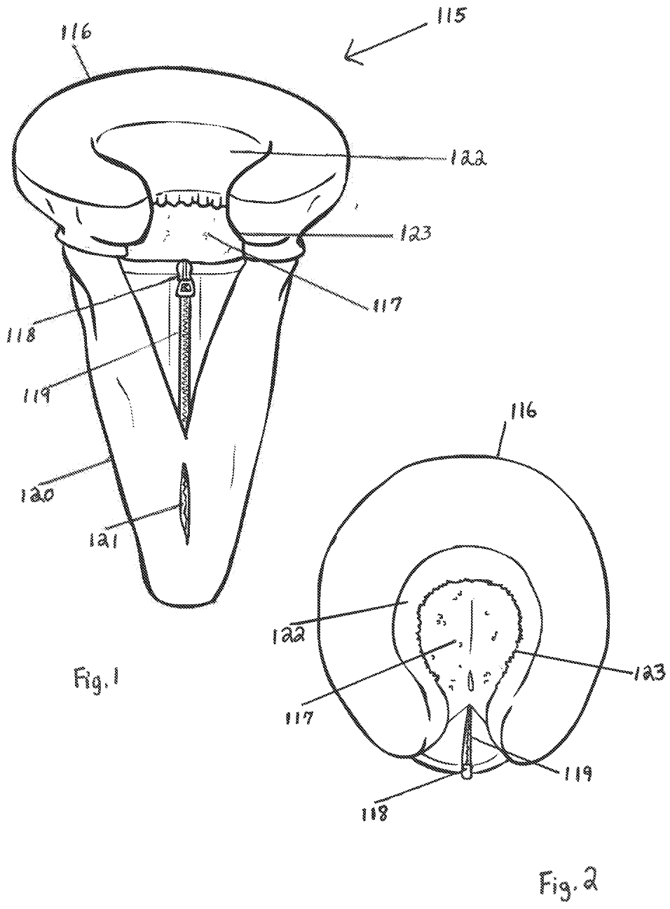

Turning now to FIG. 1 a front view of a face cradle cover with an immediate essential oil element, in accordance with an embodiment of the present invention. In preferred embodiment, the face cradle cover with immediate essential oil decongestant element 115 consists of a face cradle cover portion 116 and an immediate essential oil decongestant element 120. The face cradle cover portion 116 and the immediate essential oil decongestant permanent attachment means 123. The Immediate essential oil decongestant element is further composed of a zipper 119 and Velcro 121 portion. The immediate essential oil decongestant element 120 is connected via 123 to the face cradle cover 116 to form the face cradle cover with immediate essential oil decongestant element 115.

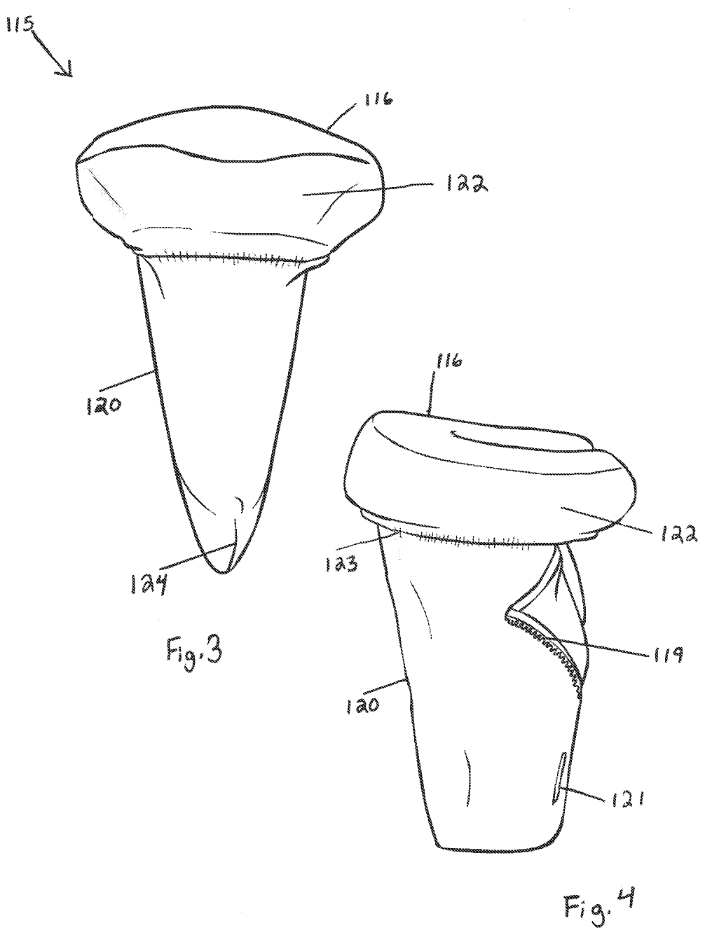

Turning now to FIG. 2 a top view of the face cradle cover 116, in accordance with an embodiment of the present invention. In a preferred embodiment, the face cradle cover consists of a permanent ¾ sewn 123 attachment to the immediate essential oil decongestant element 120. The face cradle cover 116 consists of an inside wall lining 122. FIG. 2 also reveals the immediate essential oil decongestant element interior lining 117.

Turning now to FIG. 3 is a rear back view of the face cradle cover with an immediate essential oil decongestant element 115, in accordance with an embodiment of the present invention. FIG. 3 reveals the outer rear portion of the face cradle cover 122. Fig3. also reveals the bottom seam of the immediate essential oil decongestant element 124.

Turning now to FIG. 4 which reveals a side opened view of the face cradle cover with an immediate essential oil decongestant element 115. In accordance with an embodiment of the present invention. FIG. 4 reveals the side view of the cradle covers immediate essential oil decongestant elements which comprises a top view of the cradle cover 116. Also showing its side exterior of the cradle 122. FIG. 4 reveals the connection point to the immediate essential oil decongestant element 123. FIG. 4 also reveals the side view of the zipper 118 and the side of the whole zipper partition function partition 119. Lastly FIG. 4 reveals the side view of the ¾ permanent sealed back/side portion of the immediate essential oil decongestant element to the face cradle top portion 116.

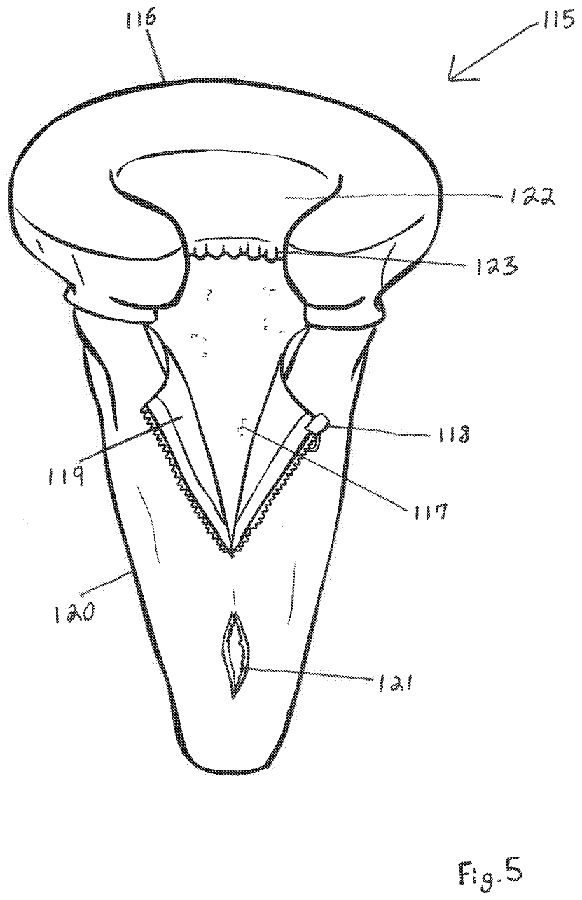

Turning now to FIG. 5 which reveals an open front view of the face cradle cover with an immediate essential oil decongestant element. In accordance with an embodiment of the present invention. FIG. 5 reveals the face rest cover with immediate essential oil decongestant element 115. FIG. 5 reveals the top of the face rest cover portion 116. FIG. 5 reveals the inside interior barrier of the face cradle cover 122, along with the connection point to the immediate essential oil elements interior lining 117. FIG. 5 lastly reveals the Velcro portion 121.

While multiple embodiments are disclosed, still other embodiments of the present invention will become apparent to those skilled in the art from this detailed description. The invention is capable of a myriad of modifications in various obvious aspects, all without departing from the spirit and the scope of the present invention. According to the drawing and description are to be regarded as illustrations in nature and not restrictive.

1. The face cradle cover with an immediate essential oil decongestant element, comprising a face cradle cover portion comprising a cover wall configured to envelop a face cradle, one or more cover walls configured to secure said cover wall to the face cradle wall. With a connection to the bottom half of the face cradle cover connected ¾ permanent stitch, wherein one portion attachment means is adapted to connect the immediate essential oil decongestant portion to the face cradle portion.

2. The face cradle cover with an immediate essential oil decongestant element of claim1 said face cradle cover is adapted to serve as a protective barrier for exposed portions of a massage table any attachments such as to form a face cradle envelope to cover the face cradle insert.

3. The face cradle cover with immediate essential oil decongestant element of claim 1, is further adapted to store essential oil drops to tissue paper (e.g. cotton balls, medical pads) to the hanging “drum-like” portion when hanging directly below the face cradle cover portion.

4. The face cradle cover with immediate essential oil decongestant element of claim 1, wherein said one or more cover wall securing means is selected from a group of cover wall securing means comprising an elastic element, a zipper, and Velcro partition.

5. The face cradle cover with an immediate essential oil decongestant element comprises a face cradle portion which is configured with cover walls to secure and envelop the face cradle insert itself. The face cradle cover element's bottom half is secured via stitch to the top half of the immediate essential oil element via (¾ semi-circle sewn stitch).

6. The face cradle cover with immediate essential oil decongestant element of claim 5, wherein said the face cradles connection point to the immediate essential oil decongestant element is created by stitch.

7. The face cradle cover with immediate essential oil decongestant element of claim 5, wherein one connection point is stitched connecting between said immediate essential oil decongestant element and the said face cradle portion.

8. The face cradle cover with immediate essential oil decongestant element of claim 5, wherein said the face cradle cover is adapted to envelop the face cradle cover.

9. The face cradle cover with immediate essential oil decongestant element of claim 5, wherein said consist of one or more cover wall securing wall means to the face cradle cover portion.

10. The face cradle cover with immediate essential oil decongestant element of claim 5, wherein said the immediate essential oil decongestant element consists of an interior lining.

11. The face cradle cover with immediate essential oil decongestant element of claim 5, wherein said interior lining is constructed from protective material, such as terry cloth or other lead industry fabric materials.

12. The face cradle cover with immediate essential oil decongestant element of claim 5, the face cradle cover and immediate essential oil decongestant elements exterior lining is constructed of protective materials.

13. The face cradle cover with immediate essential oil decongestant element of claim 5, the protective materials utilized for its construction are cotton, or other lead industry fabrics.
